(19) United States
(12) Patent Application Publication
(10) Pub. No.: US 2006/0122267 A1
(43) Pub. Date: Jun. 8, 2006

(54) Compositions and methods for attenuating mitochondria-mediated cell injury

(76) Inventors: Paul S. Brookes, Rochester, NY (US); Shey-Shing Sheu, Pittsford, NY (US); Marion W. Anders, Pittsford, NY (US)

Correspondence Address: Jane Massey Licata;Licata & Tyrrell P.C., 66 E. Main Street, Marlton, NJ, 08053, US

(21) Appl. No.: 11/316,618

(22) Filed: Dec. 20, 2005

Related U.S. Application Data
(63) Continuation-in-part of application No. PCT/US04/39739, filed on Nov. 26, 2004.
(60) Provisional application No. 60/524,833, filed on Nov. 25, 2003.

(51) Int. Cl. A61K 31/21

(52) U.S. Cl. 514/509

(57) ABSTRACT

The present invention relates to a S-nitrosated mitochondria-targeted thiol-based antioxidant prodrug and uses therefore for the prevention or treatment of diseases or conditions associated with mitochondrial dysfunction resulting from changes in the mitochondrial redox environment. When activated, prodrug of the present invention can specifically provide a NO^{•} donor and a thiol-based antioxidant to mitochondria thereby decreasing the degree of mitochondrial dysfunction.

### INTRODUCTION

This application is a continuation-in-part of PCT/US2004/039739 filed Nov. 26, 2004, which claims benefit of U.S. Provisional Patent Application Ser. No. 60/524,833, filed Nov. 25, 2003, the contents of which are incorporated herein by reference in their entirety. This invention was made in the course of research sponsored by the National Institutes of Health (Grant No. RO1 HL71158). The U.S. government may have certain rights in this invention.

### BACKGROUND OF THE INVENTION

One of the many cellular reactions of nitric oxide (NO^{•}) is S-nitrosation of thiols, resulting in the generation of S-nitrosothiols (Stamler, et al. (2001) Cell 106:675-83; Miersch & Mutus (2005) Clin. Biochem. 38:777-791). S-nitrosothiol formation has been implicated in several physiologic and pathologic phenomena, including caspase inhibition (Mannick, et al. (1999) Science 284:651-4), transport of NO^{•} in the bloodstream (Stamler, et al. (1992) Proc. Natl. Acad. Sci. USA 89:7674-7), and effects on mitochondria (Borutaite, et al. (2000) Biochim. Biophys. Acta. 1459:405-12; Hsu, et al. (2005) J. Neurochem. 92:1091-103; Zhang, et al. (2005) Am. J. Physiol. Cell Physiol. 288:840-9). Notably, formation of S-nitrosothiol has been found under hypoxic conditions (Ng, et al. (2004) Circ. Res. 94:559-565) and it is thought that some of the cardioprotective effects nitrite (Duranski, et al. (2005) J. Clin. Invest. 115:1232-1240) may be mediated through S-nitrosothiol formation.

The mitochondrion is an essential organelle for normal cellular function, being the chief site of ATP synthesis and an integrator for apoptotic signaling (Duchen (2004) Mol. Aspects Med. 25:365-451; Skulachev (1999) Mol. Aspects. Med. 20:139-84). Mitochondria interact with NO^{•} at several levels, and one particularly well-characterized example is the inhibition of complex IV (cytochrome c oxidase), via binding of NO^{•} to its binuclear Cu_{B}/heme-a₃ active site (Cleeter, et al. (1994) FEBS Lett. 345:50-4; Palacios-Callender, et al. (2004) Proc. Natl. Acad. Sci. USA 101:7630-5). Several studies have suggested that complex IV is not the only site within the respiratory chain that can be inhibited by NO^{•} (Hsu, et al. (2005) supra; Brookes, et al. (2002) Methods Enzymol. 359:305-19; Poderoso, et al. (1996) Arch. Biochem. Biophys. 328:85-92), thereby alluding to additional NO^{•}-dependent control points within mitochondria.

There are several reasons why S-nitrosation may be an important mitochondrial regulatory mechanism. Mitochondria contain sizeable thiol pools, are abundant in transition metals, and have an internal alkaline pH, all of which are known to modulate S-nitrosothiol biochemistry (Foster & Stamler (2004) J. Biol. Chem. 279:25891-7). In addition, mitochondria are highly membranous and sequester lipophilic molecules such as NO^{•}, and the formation of the putative S-nitrosating intermediate N₂O₃ is enhanced within membranes (Bruckdorfer (2005) Mol. Aspects Med. 26:3-31).

Previous studies have suggested that S-nitrosothiols may affect various parts of the respiratory chain. In particular, indirect evidence exists for S-nitrosation of complex I, the primary point of electron entry to the chain (Borutaite, et al. (2000) supra; Hsu, et al. (2005) supra; Brown & Borutaite (2004) Biochim. Biophys. Acta 1658:44-9; Clementi, et al. (1998) Proc. Natl. Acad. Sci. USA 95:7631-6). In these studies complex I inhibition upon exposure to NO^{•} was reversed by S-nitrosothiol-degradative processes, such as exposure to light, or low molecular weight thiols including glutathione (GSH) and dithiothreitol (DTT). Although this provided evidence that complex I was a target for S-nitrosation, it is notable that NO^{•} inhibition of complex IV is also sensitive to light (Sarti, et al. (2003) Free Radic. Biol. Med. 34:509-20), and there has been no direct measurement of S-nitrosothiol formation within complex I, or determination of which peptides are S-nitrosated.

The analysis disclosed herein provides the first direct evidence for S-nitrosation of mitochondrial complex I, and highlights a potential role for this protein modification in protecting mitochondria and cells from injury.

### SUMMARY OF THE INVENTION

The present invention is an S-nitrosated mitochondria-targeted thiol antioxidant. A pharmaceutical composition containing the instant prodrug in admixture with a pharmaceutically acceptable carrier is provided, as is the use of the S-nitrosated mitochondria-targeted thiol antioxidant prodrug in methods for delivering nitric oxide to mitochondria of a cell, decreasing mitochondrial dysfunction resulting from changes in the mitochondrial redox environment, and preventing or treating a disease or condition associated with mitochondrial dysfunction.

### BRIEF DESCRIPTION OF THE DRAWINGS

FIG. 1 shows that a mitochondrial-targeted NO^{•} donor protects cardiomyocytes from ischemia-reperfusion (IR) injury. Isolated adult rat cardiomyocytes were subjected to 1 hour ischemia (anoxic, glucose free medium, pH 6.5) followed by 30 minutes reoxygenation (labeled “HR”). Cell viability was assayed by Trypan blue exclusion. Ischemic preconditioning (IPC), or inclusion of 100 μM S-nitrosoglutathione (GSNO, a non-specific NO^{•} donor) led to significant protection, but a lower dose of S-nitrosoglutathione (10 μM) afforded no protection. In contrast, doses of 10 μM or 20 μM of the putative mitochondrially targeted NO^{•} donor S-nitroso-2-mercaptopropylglycine (SNO-MPG) gave dose-dependent and significant protection, with the higher dose being more effective than IPC. All data are means±SEM, N>3.

FIG. 2 shows that a mitochondrial-targeted NO^{•} donor protects cardiomyocytes from mitochondrial H⁺ leak. Cardiomyocyte respiration rate was measured with an oxygen electrode, and membrane potential with the fluorescent dye TMRE (20 nM) in a fluorimeter. H⁺ leak is essentially the amount of work the respiratory chain must do to maintain a given membrane potential, and experimentally is derived by dividing respiration by TMRE fluoresence. These data show that doses of S-nitrosoglutathione (GSNO) and S-nitroso-2-mercaptopropylglycine (SNO-MPG) that protect cardiomyocyte viability also prevent large increases in H⁺ leak. All data are means±SEM, N>3.

FIG. 3 shows the effects of S-nitroso-2-mercaptopropylglycine (SNO-MPG) on recovery from ischemia-reperfusion injury in perfused rat hearts. Hearts were perfused in constant flow mode, with Krebs-Henseleit (KH) buffer, gassed with 95/5 O₂/CO₂. Following equilibration (during which S-nitroso-2-mercaptopropylglycine was infused at 10 μM final concentration), global ischemia was imposed for 25 minutes, followed by 30 minutes of reperfusion. Cardiac functional parameters were measured throughout, with a left-ventricular balloon attached to a pressure transducer. Recovery of rate pressure product (RPP, i.e., left ventricular developed pressure multiplied by heart rate) was greater in S-nitroso-2-mercaptopropylglycine treated hearts. Control hearts in these experiments were subject to dark conditions.

FIG. 4 shows the effects of light on recovery from ischemia-reperfusion injury in rat hearts. Hearts were perfused in the dark (complete darkness, all lights off for the entire procedure), or the light (ambient fluorescent laboratory lights ˜2 meters above the heart position on the perfusion apparatus).

### DETAILED DESCRIPTION OF THE INVENTION

Mitochondrial dysfunction, primarily mediated by Ca²⁺ overload and reactive oxygen species plays a key pathologic role in ischemia-reperfusion injury. Nitric oxide (NO^{•}) exhibits some protective effects against ischemia-reperfusion injury, but also has pleiotropic cell signaling actions including specific reactions with protein heme groups, protein thiols, and other radicals, such that the therapeutic efficacy of global NO^{•} donors is limited. It has now been found that an S-nitrosated mitochondria-targeted thiol-based antioxidant prodrug, when activated, can specifically provide both NO and a thiol-based antioxidant to mitochondria, thereby decreasing the degree of mitochondrial dysfunction resulting from changes in the mitochondrial redox environment. As disclosed herein, S-nitrosation of mitochondrial complexes by a NO^{•} donor, is inhibitory. Such reversible inhibition can decrease mitochondrial reactive oxygen species generation, and limit mitochondrial Ca²⁺ overload. As such, the prodrugs of the instant invention find application in the prevention and treatment of diseases or conditions associated with mitochondrial dysfunction resulting from changes in the mitochondrial redox environment, in particular those diseases or conditions resulting from excessive reactive oxygen species production and/or mitochondrial Ca²⁺ overload. (e.g., ischemia-reperfusion injury and related pathologies).

To demonstrate that low molecular weight S-nitrosothiols can interact with complex I and modify it at the molecular level by S-nitrosation, it was investigated whether S-nitrosoglutathione treatment affected complex I activity (rotenone-sensitive NADH/Co-Q1 oxidation/reduction). As previously shown (Borutaite, et al. (2000) supra; Hsu, et al. (2005) supra) S-nitrosoglutathione treatment resulted in complex I inhibition that was reversible by exposure of mitochondria to light, indicative of an S-nitrosation-dependent mechanism.

Thus, methodologies were developed to measure S-nitrosation within complex I. A biotin-switch analysis (Jaffrey & Snyder (2001) Sci. STKE 2001(86):PL1) was performed on the entire mitochondrial protein extract to demonstrate the nitrosation capabilities of a variety of NO^{•} donors including S-nitrosoglutathione, peroxynitrite (ONOO⁻), and DETA-NONOate. The results of this analysis showed that S-nitrosoglutathione resulted in far more S-nitrosation than DETA-NONOate or ONOO⁻. This and other studies (Poderoso, et al. (1996) supra; Steffen, et al. (2001) Biochem. J. 356:395-402) indicate that S-nitrosoglutathione is a useful agent for the study of mitochondrial protein S-nitrosation. The dose response of mitochondrial protein S-nitrosation in response to S-nitrosoglutathione was also determined over the range 50-250 μM, with a plateau of S-nitrosation reached at ˜200 μM S-nitrosoglutathione.

Since the biotin-switch assay has a limited linear dynamic range and only detects protein-bound S-nitrosothiols, a chemiluminescent NO analysis over a wider range of S-nitrosoglutathione concentrations (10 μM to 1 mM) was also performed. The results showed that as S-nitrosoglutathione treatment concentration increased, the total S-nitrosothiol (i.e., free plus protein-bound) increased in a classical hyperbolic manner, approaching saturation at ˜300 μM S-nitrosoglutathione. Together, these data indicate that formation of protein bound S-nitrosothiol saturates at ˜250 μM S-nitrosoglutathione. At concentrations above 250 μM, secondary reactions such as free S-nitrosothiol or NO₂⁻ formation were observed. These data in isolated mitochondria agree with similar observations in which whole cells were treated with S-nitrosoglutathione (Gordge, et al. (1998) Biochem. Pharmacol. 55:657-65).

Efforts to detect S-nitrosation within mitochondrial complex I itself relied on isolating the complex using a modified blue-native gel electrophoresis method, followed by chemiluminescent S-nitrosothiol analysis. Alterations to the blue-native gel method to preserve S-nitrosothiol (metal chelators, low voltage, dark) did not affect the pattern of respiratory complex separation (Brookes, et al. (2002) Proteomics 2:969-77). Chemiluminescent analysis of excised gel bands gave an S-nitrosothiol profile of the gel, with four major peaks. The first of these was in a gel segment enriched in complex I.

Dose responses of complex I S-nitrosation and inhibition to S-nitrosoglutathione (10-500 μM) were also determined. Assuming heart mitochondria contain ˜50 pmol complex I per mg protein (Schagger & Pfeiffer (2001) J. Biol. Chem. 276:37861-37867), it was observed that S-nitrosation plateaud at a ˜7:1 mol/mol ratio (i.e., 7 mols S-nitrosothiol per mol complex I), while maximum complex I inhibition was ˜20-25%. This apparent mis-match between the stoichiometry of S-nitrosation (7:1) and the degree of inhibition (25%) may reflect S-nitrosation of complex I on multiple subunits. Alternatively, since the spectrophotometric complex I activity assay was performed at 340 nm, close to the absorption maxima of S-nitrosothiols (335 nm), S-nitrosothiols within complex I may have been destroyed during the assay, leading to underestimation of inhibition by S-nitrosation. Nevertheless, a link between S-nitrosation and inhibition was demonstrated by a linear correlation (r²=0.86) between these parameters. In addition, these data were quantitatively consistent with chemiluminescent data on total S-nitrosothiol content (supra). With 100 μM S-nitrosoglutathione treatment, total S-nitrosothiol was ˜1700 pmol/mg mitochondrial protein, with ˜20% (350 pmols) originating from complex I. Similarly, at a 7:1 molar ratio of S-nitrosation, 1 mg mitochondrial protein contains 50 pmol complex I (Schagger & Pfeiffer (2001) supra), and thus 7×50=350 pmols S-nitrosothiol.

To simplify separation of complex I, SUPEROSE™ 6 gel filtration chromatography was employed as a liquid phase high-throughput separation method (Danial, et al. (2003) Nature 424:952-6). Chemiluminescent analysis of column fractions was then used to construct an S-nitrosothiol profile. Most S-nitrosothiol concentrated in two regions of the chromatogram, eluting at ˜35% and 85% of the column bed volume. There was no correlation between S-nitrosothiol and protein contents of the fractions (r²=0.33), indicating that S-nitrosothiol peaks were not simply fractions containing the most protein.

The fraction with most S-nitrosothiol content was enriched in complex I activity, and was then further analyzed by biotin switch assay to identify S-nitrosated peptides (i.e., which of the 46 subunits in complex I was S-nitrosated). The data indicated that a single peptide within this fraction was S-nitrosated. Analysis by peptide mass fingerprinting (MALDI-TOF) identified this protein as the 75 kDa subunit of complex I (GENBANK Accession No. 51858651). The MOWSE score for the excised protein was 76 (>58 significant, p<0.05) with 33% sequence coverage.

Together, the S-nitrosothiol content analysis and S-nitrosothiol target identification, revealed that ˜20% of the total protein-bound S-nitrosothiol in S-nitrosoglutathione-treated mitochondria was contained in a single peptide, the 75 kDa subunit of complex I. There are ˜2000 proteins in mitochondria, and >95% of all proteins have at least one cysteine. Therefore the presence of such a large fraction of S-nitrosothiol within this single peptide indicates it is a highly specific S-nitrosation target. Furthermore the 7:1 stoichiometry of S-nitrosation indicates that multiple cysteine residues within this peptide are S-nitrosated.

Reversible inhibition of complex I by S-nitrosation may represent an additional mechanism for NO^{•}-dependent control of the mitochondrial respiratory chain. Studies have suggested that sites other than complex IV within the chain may be targets for NO^{•} (Brookes, et al. (2002) supra; Poderoso, et al. (1999) J. Biol. Chem. 274:37709-16). The relative contributions of complex IV heme-nitrosylation vs. complex I S-nitrosation can be determined, and the balance between these two events may shift depending on the intra-mitochondrial conditions (pH, O₂ tension, etc.)

The instant data have important implications for the effect of NO^{•} on mitochondrial reactive oxygen species generation. Inhibition of complex IV by NO^{•} can cause back-up of electrons in the respiratory chain and increase reactive oxygen species generation at complex III (Poderoso, et al. (1996) supra; Brookes & Darley-Usmar (2002) Free Radic. Biol. Med. 32:370-4). However, because complex I is an entry point for electrons into the chain, reversibly inhibiting it by S-nitrosation would lower the electron flux through the chain, thereby lowering reactive oxygen species generation. While complex I inhibition may increase reactive oxygen species at the complex itself (Taylor, et al. (2003) J. Biol. Chem. 278:19603-10), it should be noted that complex I is quantitatively a much smaller source of reactive oxygen species than complex III (Chen, et al. (2003) J. Biol. Chem. 278:36027-31). Thus, overall a small S-nitrosothiol-induced increase in complex I reactive oxygen species can be beneficial by inhibiting large-scale reactive oxygen species generation at complex III.

To demonstrate a beneficial role for mitochondrial S-nitrosation, mitochondria were isolated from hearts subjected to ischemic preconditioning. In ischemic preconditioning, short periods of nonlethal ischemia protect the heart against subsequent ischemia-reperfusion injury (Murry, et al. (1986) Circulation 74:1124-1136). In addition, significant roles for both NO^{•} and mitochondria in ischemic preconditioning have been proposed (Zaugg & Schaub (2003) J. Muscle Res. Cell Motil. 24:219-249). In this regard, S-nitrosothiol was undetectable (limit 0.5-1 pmol) in control mitochondria, but easily detected in ischemic preconditioned mitochondria (16 pmols SNO/mg mitochondrial protein). While this S-nitrosothiol signal was not assigned to complex I, the instant data indicates that complex I S-nitrosation is significant whenever mitochondrial S-nitrosothiol are present. In addition, while the exact amount of mitochondrial S-nitrosothiol detected in ischemic preconditioning was much lower than that seen following treatment of mitochondria with S-nitrosoglutathione, significant S-nitrosothiol degradation during the >1 hour mitochondrial isolation procedure, especially at the homogenization step, was seen. The source of mitochondrial S-nitrosothiol during ischemic preconditioning is not known, but it may originate from nitrite (Duranski, et al. (2005) supra), which indicates that complex I S-nitrosation may underlie some of the cardioprotection mediated by nitrite.

Having demonstrated that a non-mitochondria-targeted NO^{•} donor (i.e., S-nitrosoglutathione) can S-nitrosate complex I thereby inhibiting its activity, S-nitrosation of mitochondria by an S-nitrosated mitochondria-targeted thiol-based antioxidant prodrug, namely S-nitroso-2-mercaptopropylglycine, was determined. At the same concentrations (i.e., 10-100 μM), S-nitroso-2-mercaptopropylglycine resulted in a much greater degree of S-nitrosation of mitochondrial proteins than did S-nitrosoglutathione. Because the only components in this treatment system were mitochondria and the NO donor, and the assay detects protein s-nitrosation on both sides of the mitochondrial membrane (inside and outside), no differences in S-nitrosation between these two agents were expected. Therefore, the fact that a difference was detected highlights that targeting of the NO^{•} donor to the mitochondria can yield specific S-nitrosation patterns.

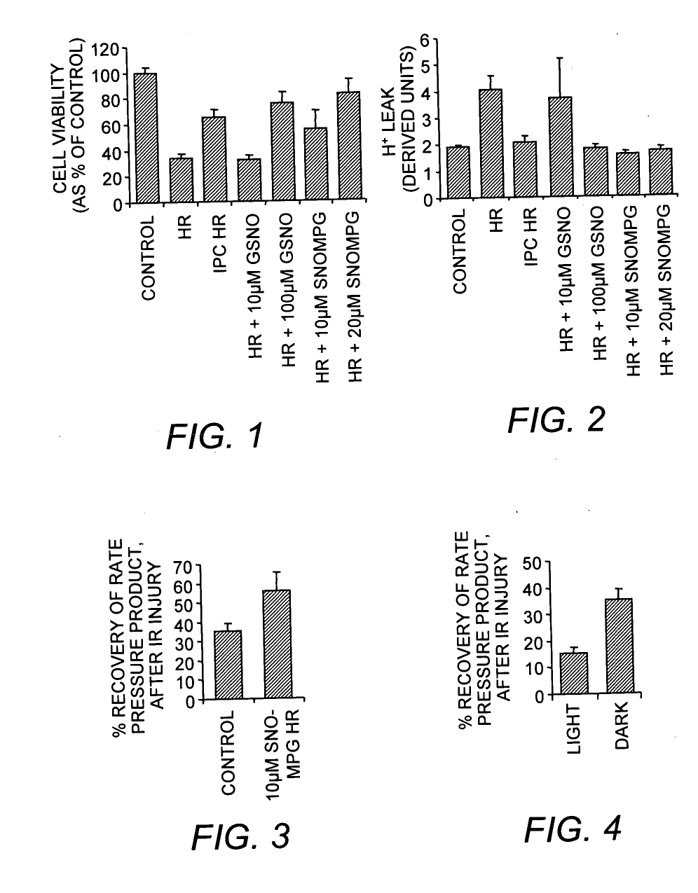

To demonstrate the therapeutic efficacy of a S-nitrosated mitochondria-targeted thiol-based antioxidant prodrug of the present invention, S-nitroso-2-mercaptopropylglycine was analyzed in an adult rat cardiomyocyte model of ischemia-reperfusion injury. Unexpectedly S-nitroso-2-mercaptopropylglycine, when used at concentrations 10-20 μM, provided significant protection of cardiomyocytes from ischemia-reperfusion injury (FIG. 1). To achieve comparable levels of protection, 100 μM of the classical NO^{•} donor S-nitrosoglutathione was required. One of the hallmarks of mitochondrial damage in ischemia-reperfusion injury is an increase in mitochondrial H+ leak (the permeability of the mitochondrial inner membrane to protons). S-nitroso-2-mercaptopropylglycine was also found to protect against the large increase in proton leak induced by ischemia-reperfusion injury (FIG. 2).

Similarly, in a Langendorff perfused rat heart model of ischemia-reperfusion injury, S-nitroso-2-mercaptopropylglycine protected the perfused heart from ischemia-reperfusion injury. The data in FIG. 3 show that perfusion of the rat heart with 10 μM S-nitroso-2-mercaptopropylglycine for 20 minutes prior to ischemia led to increased recovery of cardiac function after ischemia-reperfusion.

During the course of conducting experiments to detect S-nitrosothiols in perfused hearts, it became necessary to perform experiments in the dark. In doing so, it was unexpectedly discovered that ambient fluorescent laboratory light had a detrimental effect on recovery of the heart from ischemia-reperfusion injury. As shown in FIG. 4, the simple difference of switching the laboratory lights on or off caused a significant difference in recovery of heart function after ischemia-reperfusion injury. The spectrum of light under the ambient fluorescent laboratory light conditions, obtained with an Ocean Optics spectrometer, indicated major peaks at approximately, 405 nm, 440 nm, 490 nm, 545 nm, 590 nm, and 610 nm. These results are consistent with the ability of endogenous, light-sensitive S-nitrosothiols in protecting the heart from ischemia-reperfusion injury.

Having demonstrated the use of an S-nitrosated mitochondria-targeted thiol-based antioxidant prodrug to provide a NO^{•} donor and a thiol-based antioxidant to mitochondria, thereby protecting cells from ischemia-reperfusion injury, the present invention embraces S-nitrosated mitochondria-targeted thiol-based antioxidant prodrugs and uses thereof.

As used in the context of the present invention, a prodrug is a compound that undergoes biotransformation via a metabolic process or characteristics of the cellular environment before exhibiting its pharmacological effects. This can include chemical transformation in a unique sub-cellular environment such as the mitochondrial matrix, that is not dependent on a specific enzymatic activity. Prodrugs are generally viewed as drugs containing specialized non-toxic protective groups used in a transient manner to alter or to eliminate undesirable properties in the parent molecule until the target site is reached. Advantageously, the alkaline pH and presence of the low molecular weight thiol gluthatione in the mitochondrial matrix both facilitate the biotransformation or activation of the instant prodrug to release a NO^{•} donor and a thiol-based antioxidant into mitochondria.

The term antioxidant, as used in the context of the instant invention, refers to a compound that when present at low concentrations compared to those of an oxidizable substrate significantly delays or prevents oxidation of that substrate. There is an abundance of oxidizable substrates in the cell, including proteins, lipids, carbohydrates, and DNA. Thus antioxidants can function to prevent the formation of (or detoxify) free radicals, and to scavenge reactive oxygen species (e.g., superoxide, hydrogen peroxide, hypochlorous acid, ozone, singlet oxygen, hydroxyl radical, and peroxyl, alkoxyl, and hydroperoxyl radicals) or their precursors.

For the purposes of the present invention, mitochondria-targeting of the instant prodrugs is achieved by selecting thiol antioxidants which can be transported across mitochondrial membranes by transport systems, e.g., the well-known choline transporters (Apparsundaram, et al. (2000) Biochem. Biophys. Res. Commun. 276:862-867; Okuda, et al. (2000) Nat. Neurosci. 3:120-125; Porter, et al. (1992) Biol. Chem. 267:14637-14646), carnitine acetyltransferase or dicarboxylate transporter; or the mitochondrial electrochemical potential gradient which concentrates hydrophilic, positively-charged choline esters, N-heterocycle esters, carnitine esters, or choline amides of glutathione or other peptide-based or amino acid-based antioxidants in mitochondria.

In this regard, particular embodiments of the present invention embrace the S-nitrosation of thiol-based antioxidants derived from amino acids, amino acid derivatives, peptides (i.e., two or more amino acids), or combinations thereof. Particularly suitable antioxidants which can be thiolated, if a thiol group is not already present, and S-nitrosated in accordance with the present invention include, but are not limited to, those disclosed in WO 2005/051978.

According to one embodiment of the present invention, the thiol antioxidant is provided as a single amino acid or amino acid derivative that possesses antioxidant activity. If the amino acid or amino acid derivative does not contain a thiol group, the amino acid or amino acid derivative can be thiolated in accordance with the methods disclosed herein. Exemplary amino acids and derivatives thereof include, without limitation, glutamic acid, cysteine, N-acetyl-cysteine, glycine, and 2,2-dialkylthiazolidine-4-carboxylic acid, N-mercapto alkanoyl cysteines, and 2-mercaptopropionylglycine; and choline ester, N-heterocycle ester, carnitine ester, and choline amide derivatives thereof.

According to another embodiment of the present invention, the thiol antioxidant is provided as two or more amino acids or amino acid derivatives, defined herein as a peptide-based antioxidant, wherein one or more of the amino acids or amino acid derivatives of the peptide possess antioxidant activity. In certain embodiments, the peptide-based antioxidant is at least two amino acids (or amino acid derivatives) in length, wherein at least one of the amino acids possesses antioxidant activity. In other embodiments, the peptide-based antioxidant moiety is from two to about ten amino acids (or amino acid derivatives) in length, wherein one or more of the amino acids possess antioxidant activity. In still further embodiments, the peptide-based antioxidant is from two to about five amino acids (or amino acid derivatives) in length, wherein one or more of the amino acids possess antioxidant activity. If one or more of the amino acids or amino acid derivative does not contain a thiol group, one or more of the amino acids or amino acid derivatives can be thiolated in accordance with the methods disclosed herein. Exemplary peptide-based antioxidants for use in accordance with the instant invention include, without limitation, L-γ-glutamylcysteine, L-γ-glutamylglycine, L-cysteinylglycine, glutathione, N-acetyl glutathione, L-carnosine, L-carnitine, and acetyl-L-carnitine; and choline ester, N-heterocycle ester, carnitine ester, and choline amide derivatives thereof.

As will be appreciated by one of skill in the art, the amino acids and their derivatives that form the thiol antioxidant can be L-amino acids or derivatives thereof, D-amino acids or derivatives thereof, or combinations thereof (e.g., in a peptide-based thiol antioxidant).

Particularly suitable compounds which can be S-nitrosated and used in accordance with the instant invention include, without limitation, carnitine and choline esters of N-acetyl glutathione, L-γ-glutamyl-L-cysteinylglycine choline ester, D-γ-glutamyl-L-cysteinylglycine choline ester, L-cysteine choline ester, L-γ-glutamyl-L-cysteine choline ester, D-γ-glutamyl-L-cysteine choline ester, N-acetyl-L-cysteine choline ester, N-acetyl-L-cysteine choline amide, glutathione choline amide, 2-dimethylthiazolidine-4-carboxylic acid, and L-2-(trimethylamino)ethyl-2,2-dimethylthiazolidine-4-carboxylic acid, [2-(2-acetylamino-3-mercaptopropionyloxy)ethyl]trimethylammonium bromide, [2-(2)-amino-3-mercaptopropionyloxy)ethyl]trimethylammonium iodide, (2-{2-[2-(4-amino-4-carboxybutyrylamino)-3-mercaptopropionylamino]acetoxy}ethyl)trimethylammonium bromide, 2-amino-3-mercapto-propionic acid-2-dimethylamino-ethyl ester and 2-mercaptopropylglycine.

In one embodiment, the S-nitrosated mitochondria-targeted thiol antioxidant prodrug is distinct from naturally occurring S-nitrosated thiol antioxidants (e.g., S-nitrosoglutathione) in that the instant prodrugs are targeted to the mitochondria (e.g., via choline ester or choline amide derivation). In particular embodiments, the S-nitrosated mitochondria-targeted thiol antioxidant prodrug of the present invention is synthetically produced. In this regard, the amino acid-based or peptide-based antioxidant parent compound can be produced using art-established methods (see, e.g., WO 2005/051978 for synthesis of an amino acid, amino acid derivative, and peptide antioxidant). In particular embodiments the parent compound (e.g., 2-mercaptopropionylglycine) is commercially available and can be further purified prior to S-nitrosation. Wherein the parent antioxidant lacks a free thiol group, standard thiolating reagents can be employed to produce a thiol antioxidant. For example, thiol groups can be introduced using sulphur chlorides (S₂Cl₂, SO₂Cl₂, SOCl₂); phosphorus pentasulphide (P₂S₅) and specialized reagents such as thiolacetic acid, Lawessons reagent and potassium thioacetate. Subsequently, the thiol antioxidant is S-nitrosated to ˜95% purity according to general method provided in Scheme 1. 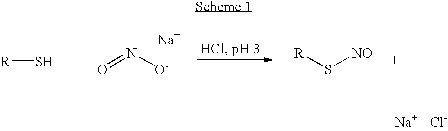
wherein R is the antioxidant.

Disappearance of the parent thiol antioxidant can be monitored according to standard assays such as the DTNB assay (Ellman's reagent), in which DTNB reacts with only free R-SH to generate a R-SH-TNB derivative which absorbs at 412 nm (ε=13600 M⁻¹). Formation of the S-nitrosated mitochondria-targeted thiol antioxidant prodrug is monitored by the characteristic S-nitrosothiol absorption spectrum (λ max 335 nm, ε=855 M⁻¹), and also by a Saville assay. The Saville is a derivative of the widely used Greiss assay for reactive nitrogen species, which relies on the specific de-nitrosation of S-nitrosothiols by HgCl₂. The resulting NO^{•} released is then quantified by reaction with sulfanilamide and naphthylethylenediamine, resulting in a chromophore with ε=50,000 M⁻¹ at 540 nm.

The prodrugs of the present invention can be in the form of a salt, desirably a pharmaceutically acceptable salt, i.e., a salt that retains the biological effectiveness and properties of the free base or free acid, and which is not biologically or otherwise undesirable. A salt is formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present invention.

The instant invention embraces a variety of structurally distinct thiol antioxidants and the disclosure of exemplary thiol antioxidants herein in no way limits the types of thiol antioxidants that could be S-nitrosated and used in accordance with the instant invention.

A S-nitrosated mitochondria-targeted thiol antioxidant prodrug of the present invention finds application in methods of decreasing the degree of mitochondrial dysfunction resulting from changes in the mitochondrial redox environment and preventing or treating a disease or condition associated with mitochondrial dysfunction. As such, prodrugs disclosed herein can be used alone or in admixture with a pharmaceutically acceptable carrier at an appropriate dose. Such pharmaceutical compositions can be prepared by methods and contain carriers which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000. A pharmaceutically acceptable carrier or vehicle, e.g., a liquid or solid filler, diluent, excipient, or solvent encapsulating material, is involved in carrying or transporting the prodrug from one organ, or portion of the body, to another organ, or portion of the body. Each carrier must be acceptable in the sense of being compatible with the other ingredients of the formulation and not injurious to the patient.

Examples of materials which can serve as pharmaceutically acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and other antioxidants can also be present in the compositions.

Typically, the pharmaceutical composition will contain from about 0.01 to 99 percent, desirably from about 20 to 75 percent of active compound(s), together with the carriers and/or excipients.

The compositions of the present invention can be administered parenterally (for example, by intravenous, intraperitoneal, subcutaneous or intramuscular injection), topically (including buccal and sublingual), orally, intranasally, intravaginally, or rectally according to standard medical practices. For example, application to mucous membranes and/or lungs can be achieved with an aerosol or nebulized spray containing small particles of a prodrug of this invention in a spray or dry powder form.

The selected dosage level will depend upon a variety of factors including the activity of the particular thiol antioxidant, the route of administration, the time of administration, the rate of excretion or metabolism of the particular compound being employed, the duration of the treatment, other drugs and/or materials used in combination with the particular antioxidant employed, the age, sex, weight, condition, general health and prior medical history of the patient being treated, and like factors well-known in the medical arts.

A physician or veterinarian having ordinary skill in the art can readily determine and prescribe the effective amount of the pharmaceutical composition required. For example, the physician or veterinarian could start doses of the prodrug and increase or decrease the levels as required in order to achieve the desired therapeutic effect. This is considered to be within the skill of the artisan and one can review the existing literature on a specific compound or similar compounds to determine optimal dosing.

The realization that acute and chronic stresses to the cells leads to structural and functional impairments of mitochondria has redefined the role of mitochondria in disease etiology. Mitochondrial dysfunction resulting from changes in the mitochondrial redox environment, e.g., ischemic-reperfusion conditions, triggers signaling cascades for necrosis and apoptosis of cells and results in organ failure and diseases. S-nitrosation of complex I is beneficial in ischemia-reperfusion, since it diminishes overall mitochondrial reactive oxygen species generation, decreases ΔΨₘ, prevents Ca²⁺ uptake, and prevents mitochondrial permeability transition pore opening (Brookes, et al. (2000) J. Biol. Chem. 275:20474-20479). Accordingly, having demonstrated that a S-nitrosated mitochondria-targeted thiol-based antioxidant prodrug of the present invention can S-nitrosate complex I and protect against an increase in mitochondrial inner membrane proton leak, thereby decreasing the degree of mitochondrial dysfunction, the instant prodrugs are useful in the prevention and treatment of a variety of diseases and conditions associated with changes in the mitochondrial redox environment.

In accordance with prevention or treatment, an effective amount of a S-nitrosated mitochondria-targeted thiol antioxidant prodrug is administered to a subject having or at risk of having a disease or condition associated with changes in the mitochondrial redox environment so that at least one sign or symptom of the disease or condition (e.g., cell necrosis and apoptosis or organ failure) is reduced, inhibited, ameliorated, or delayed. The amount administered can be dependent upon the disease to be treated, antioxidant being employed, and the pharmacokinetics and pharmacodynamics of the drug in the subject being treated. Moreover, as demonstrated herein, manipulation of light levels during treatment can augment the protective effects of the instant prodrugs. Thus, careful control of lighting conditions during the clinical use of the instant prodrugs can be used to modulate their cytoprotective efficacy, due to the known light sensitivity of the S-nitrosothiol moiety. This is particularly relevant to the surgical and perioperative use of the instant prodrugs, due to the high intensity lighting used in operating room situations.

NO^{•} effects on mitochondria are competitive with O₂, and thus enhanced at low O₂ tensions (i.e., during ischemia). Thus, particular embodiments embrace the prevention or treatment of diseases or condition resulting from ischemia-reperfusion and related pathologies. For example, the protective properties of the instant prodrug are in particular suitable for the prevention or treatment of ischemia-reperfusion injury, myocardial infarction, renal or intestinal ischemic injury, heart attack, and corresponding neuronal phenomena (e.g., stroke). Prodrugs of the invention could be applied in a preventive strategy, through long-term delivery/supplementation, in patients designated as “at-risk” for these conditions, or through acute delivery in the immediate perioperative period (e.g. elective cardiac surgery, or emergency room treatment of acute myocardial infarction). In addition, the instant prodrugs can be used in cardioplegic solutions for transplant of the heart, liver, or other organs.

Accordingly, the present invention is also a method of using the instant prodrug for decreasing the degree of mitochondrial dysfunction in a cell or tissue resulting from changes in the mitochondrial redox environment. This method of the invention involves contacting a cell or tissue (e.g., an organ to be transplanted) with an effective amount of a S-nitrosated mitochondria-targeted thiol-based antioxidant prodrug such that upon activation, the NO donor and thiol antioxidant are released from the prodrug form and decrease mitochondrial dysfunction. Effectiveness of the prodrug can be monitored using any established method. For example, protection of mitochondria from oxidative damage and apoptosis is measured by determining lipid peroxidation (thiobarbituric acid reactive species, or isoprostane measurements by mass spectrometry), cytochrome c release, caspase-3 activation, DNA fragmentation, inactivation of complex I and aconitase, expression of transferrin receptor, mitochondrial iron uptake, mitochondrial membrane potential, fluorescent measurements of mitochondrial reactive oxygen species generation, or indices of free radical mediated DNA damage such as 8-OH-guanine accumulation.

As will be readily appreciated by one of skill in the art, the antioxidative activities of the instant prodrugs may also exhibit some antioxidative activities in the cytoplasm in the prodrug form, or alternatively, once activated leave the mitochondria and exert activity in the cytoplasm. Thus, while antioxidative actions primarily occur in the mitochondria, antioxidative activity is contemplated within the cellular domain from the plasmalemma through the cytoplasm, to golgi, to endoplasmic reticulum, to the mitochondria.

The invention is described in greater detail by the following non-limiting examples.

### EXAMPLE 1

### Materials

Male Sprague-Dawley rats (Rattus norvegicus), 200 gram body mass, were from Harlan (Indianapolis, Ind.). All chemicals were reagent grade from Sigma (St. Louis, Mo.), except SUPEROSE™ 6 (GE Biosciences, Piscataway, N.J.), DETA-NONOate (Alexis, San Diego, Calif.), and EZ-link biotin HPDP (Pierce, Rockford, Ill.). Peroxynitrite was synthesized via the reaction of NaNO₂ with acidified H₂O₂ according to standard methods (Moro, et al. (1994) Proc. Natl. Acad. Sci. USA 91:6702-6), was quantified spectrophotometrically (302 nm, ε=1670 M⁻¹), and stock solutions were prepared in 10 mM NaOH. S-nitrosothiols were synthesized via the reaction of RSH with acidified NaNO₂ as established in the art (Frank, et al. (1999) Biochem. J. 338 (Pt 2):367-74), precipitated using acetone, filtered and washed with ether under vacuum, freeze dried, and quantified using the Saville assay (Tarpey, et al. (2004) Am. J. Physiol. Regul. Integr. Comp. Physiol. 286:431-44). In addition, S-nitrosothiol content was monitored spectrophotometrically (338 nm, ε=855 M⁻¹).

### EXAMPLE 2

### Mitochondrial Isolation, Incubations, and Assays

Rat heart mitochondria were isolated using differential centrifugation (Tompkins, et al. (2005) Biochim. Biophys. Acta PMID: 16278076). Protein was determined by the Folin-phenol method (Lowry, et al. (1951) J. Bio. Chem. 193:265-75) against a standard curve constructed using bovine serum albumin. Mitochondrial incubations and all subsequent steps were performed in the dark. One mg of mitochondrial protein was suspended in 1 mL of mitochondrial respiration buffer (Tompkins, et al. (2005) supra) containing respiratory substrates glutamate (5 mM), malate (2.5 mM) and succinate (5 mM). Nitric oxide donors (S-nitrosoglutathione 1 mM, or DETA-NONOate 2 mM) were then added, and the suspension incubated at 37° C. for 40 minutes with periodic aeration. Samples were centrifuged (5 minutes, 14,000×g), and supernatants removed. The pellets were then resuspended in 1 mL of respiration buffer, and centrifuged again. Pellets were frozen in liquid N₂ for subsequent analysis. For ONOO⁻ treatments, 4 μL of 25 mM ONOO⁻ stock (100 μM final concentration) was added the cap of the reaction tube, the cap quickly closed and the tube shaken immediately. The process was then repeated twice at 30 second intervals, for a total of 3 additions. Mitochondria were then pelleted, washed, and frozen as for S-nitrosoglutathione samples. Complex I activity was assayed as the rotenone-sensitive oxidation of NADH, at 340 nm in the presence of coenzyme Q1 (Borutaite, et al. (2000) supra). Complex IV was assayed by monitoring the cyanide-sensitive first-order oxidation of reduced cytochrome c, at 550 nm according to established methods (Cleeter, et al. (1994) supra).

### EXAMPLE 3

### Blue-Native Gel Electrophoresis & Protein Extraction

All procedures were performed in the dark. Blue-native gel electrophoresis was carried out in accordance with standard method (Brookes, et al. (2002) supra) with minor adjustments, loading 200 μg of mitochondrial protein per well. High-blue cathode buffer contained DTPA (100 μM) and EDTA (1 mM). In addition, DTPA (100 μM) was added to the buffer used for preparing gradient gels. Gels were run at a constant 40 V for 6 hours, and cut into (˜23) 2 mm bands, which were frozen at −80° C. Prior to chemiluminescent analysis, protein extraction from the gel was performed at room temperature. Each band was homogenized in 100 μL of gel extraction buffer containing urea (6 M), phosphate-buffered saline (1×), DTPA (100 μM), EDTA (1 mM), and lauryl-maltoside (1% w/v), pH 7.2. The homogenate was centrifuged at 2000×g at room temperature for 2 minutes. The supernatant was removed and placed in a clean tube on ice. Additional buffer was added to the gel pieces, and the homogenization/centrifugation process repeated three more times, yielding a final extracted sample of ˜300 μL volume.

### EXAMPLE 4

### SUPEROSE™ 6 Column Chromatography

A 25 cm×1 cm BIO-RAD® glass ECONO-COLUMN™ was used, with appropriate flow adaptor (BIO-RAD®, Hercules Calif.) and a MASTERFLEX™ peristaltic pump (Cole Parmer, Vernon Hills, Ill.). Mitochondrial protein (3 mg) was extracted in 300 μL of column running buffer containing Tris (50 mM), KCl (50 mM), DTPA (100 μM), and lauryl-maltoside (1% w/v), pH 7.7. The sample (250 μL) was loaded onto the column, which was run at a flow rate of 1 mL/minute. Fractions (200 μL) were collected, and their A₂₈₀ measured to determine relative protein content. In addition, the S-nitrosothiol content and complex I activity of each fraction were analyzed as disclosed herein. Chromatography experiments were performed over several months, with slight variations in the column dimensions. Therefore, for data comparison the relative position of eluted column fractions was expressed as a percentage of the total column bed volume. For example if the column had a bed volume of 20 mL, then a fraction at 1 mL elution volume would correspond to 5% of the bed volume. Within a given data set the S-nitrosothiol, protein, and complex I activity profiles were all internally consistent.

### EXAMPLE 5

### Chemiluminescent and Biotin-Switch Analysis of S-nitrosothiol Content

Chemiluminescent analysis of S-nitrosothiol content was performed on samples originating from blue-native gels, intact mitochondria and SUPEROSE™ 6 chromatography fractions, according to known methodologies (Yang, et al. (2003) Free Radic. Res. 37:1-10). Blue-native samples (300 μL) were divided among three tubes to undergo preparatory chemistry which included the addition of either 40 μL extraction buffer, 20 μL of buffer plus 20 μL 5% sulfanilamide, or 20 μL 5% sulfanilamide plus 20 μL 50 mM HgCl₂. Samples of intact mitochondria (1 mg pellets) were solubilized in 300 μL of extraction buffer and subjected to the same chemistry. Fractions originating from SUPEROSE™ 6 chromatography (200 μL) were divided among three tubes (50 μL), to which was added either 10 μL column running buffer, 5 μL of 5% sulfanilamide plus 5 μL buffer, or 5 μL 5% sulfanilamide plus 5 μL 50 mM HgCl₂. All samples were reacted for 2 minutes, then 50 μL of each injected into an argon-fed purge vessel containing tri-iodide reagent (Yang, et al. (2003) supra), connected to a Seivers NOA 280 chemiluminescent NO^{•} analyzer (Ionics Instruments, Boulder, Colo.). Each sample was run in duplicate and quantified using a standard curve created with known concentrations of NaNO₂.

For biotin-switch analysis, intact mitochondrial pellets (1 mg protein solubilized in extraction buffer) or chromatography column fractions were analyzed in accordance with known methods (Jaffrey & Snyder (2001) supra). Samples were separated in duplicate on non-reducing SDS-PAGE gels; half the gel was stained with COOMASSIE® blue, and the other half blotted and probed with streptavidin-HRP and ECL™ detection (GE biosciences). Blots and stained gels were aligned, and S-nitrosated proteins excised, trypsinized, and identified by peptide mass fingerprinting. Mass spectra (MALDI-TOF) were analyzed using the MASCOT™ algorithm available on the world-wide web at matrixscience.com.

### EXAMPLE 6

### Heart Perfusions and Mitochondrial Isolation

Langendorff heart perfusions were performed as described (Tompkins, et al. (2005) supra) in constant flow mode, with the exception that all steps, including subsequent mitochondrial isolations, were performed in the dark. Hearts were preconditioned by subjecting them to three 5 minute periods of global ischemia, with 5 minute reperfusions in between. Control hearts were continuously perfused for 30 minutes. At the end of perfusion protocols, hearts were removed and placed in ice-cold buffer and mitochondria prepared as described herein.

### EXAMPLE 7

### Isolated Perfused Heart Model of Ischemia-Reperfusion Injury

Rat hearts were perfused according to methods well-known in the art (Digerness, et al. (2003) J. Thorac. Cardiovasc. Surg. 125:863-871), with Krebs-Henseleit (KH) buffer, gassed with 95/5 O₂/CO₂. Following equilibration (during which test agents were infused at 10-20 μM), global ischemia was imposed for 25 minutes, followed by 30 minutes of reperfusion. Cardiac functional parameters (left ventricular pressure, rate pressure product, oxygen consumption, diastolic stiffness, etc.) were measured throughout, and mitochondria were isolated at the end of the procedure. Several mitochondrial parameters are then determined including respiration, Ca²⁺ loading capacity, reactive oxygen species generation, permeability transition pore opening threshold, respiratory complex activities (Brookes, et al. (2002) Methods Enzymol. 359:305-319), and protein S-nitrosation as disclosed herein.

### EXAMPLE 8

### Cardiomyocyte Model of Ischemia-Reperfusion Injury

Adult rat cardiomyocytes were prepared by collagenase perfusion according to standard methods (Dai, et al. (2001) supra), yielding ˜4×10⁶ cells per heart, >80% rod-shaped and viable. Incubations were in a shaking water bath at 37° C., each utilizing 5×10⁵ cells in 5 mL of the KH buffers described below. For the control, cells were incubated for 2 hours in oxygenated KH buffer (95/5 O₂/CO₂). For ischemia-reperfusion treatment, cells were subjected to 30 minutes in oxygenated KH buffer, 1 hour in “ischemic” KH buffer (95/5 N₂/CO₂, pH 6.5, no glucose), and 30 minutes in oxygenated KH buffer. For the ischemia-reperfusion injury and test compound group of cells, ischemia-reperfusion injury was carried out as described above with test compound present during the 30 minute equilibration prior to ischemia-reperfusion and absent during ischemia. Compounds were tested at concentrations ≦10 μM. At the end of each protocol, cell viability was assayed (Trypan blue), and mitochondrial function determined (measurement of ΔΨₘ with the fluorescent probe TMRE, respiration with an O₂ electrode, and S-nitrosothiol content by chemiluminescence and biotin-switch.

### EXAMPLE 9

### In vivo Mouse Model of Ischemia-Reperfusion Injury

An in vivo mouse model of ischemia-reperfusion injury provides data pertaining to long-term effects of ischemia-reperfusion on the myocardium, and demonstrates the protective effects of mitochondria-targeted prodrugs of the instant invention. The use of such a model is well-known in the art (Shishido, et al. (2003) Circulation 108:2905-2910) with ischemia-reperfusion carried out by occluding the left anterior descending coronary artery (LAD) for 45 minutes, followed by 24 hours of reperfusion. End points measured include infarct size, in addition to isolation of mitochondria from cardiac tissue, and measurement of mitochondrial functional parameters. Mitochondrial-targeted prodrugs are administered via bolus IV injection 1 hour prior to the LAD occlusion protocol, at an initial dose of 0.2 mg/kg, which is equivalent to a plasma concentration of 14 μM based on established mouse toxicology models (Diehl, et al. (2001) J. Appl. Toxicol. 21:15-23).

### EXAMPLE 10

### Statistics

Analysis of blots, gels and chromatographs are representative of at least 3 independent experiments. Other data are presented as mean±SEM, N>3. Statistical differences between control and S-nitrosoglutathione treatment groups were determined by Student's t test.

### EXAMPLE 11

### Preparation of S-NitrosoGlutathione Choline Ester

The starting material for preparation of S-nitroso-glutathione-choline ester 1 is glutathione-choline ester.

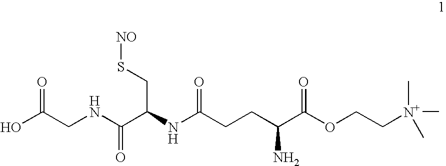

To S-nitrosate glutathione-choline ester, a trans-nitrosating column is employed. Such a strategy has been applied to trans-nitrosate low molecular weight thiols (Liu, et al. (1998) J. Pharmacol. Exp. Therap. 284:526-534), however, the beads used only had a capacity of 3 μmols free thiol per gram. As an alternative, a PS-thiophenol resin can be employed (Argonaut Technologies, San Carlos, Calif.), which has a capacity of 1.5 mmols free thiol per gram. When swollen (7 mL/gram bed volume), this resin affords a concentration of 0.2 M free thiol.

All steps are performed at room temperature in subdued light. The resin is swollen in DMF and packed into a disposable plastic column, then reduced by adding a solution of 1 M DTT in DMF. The column is capped, shaken for 5 minutes to agitate the resin and washed with several volumes of DMF to elute excess DTT. An aliquot of resin is removed and subjected to a DTNB assay to quantitate residual free thiol. A solution of 100 mM NaNO₂ is prepared in 2 M HCl in DMF, and immediately added to the column, which is then capped and shaken for 5 minutes at room temperature. The solution is then eluted and the column washed with several volumes of DMF to remove excess nitrosating agents. The presence of S-nitrosothiol is confirmed by a pink coloration of the resin, characteristic of S-nitrosothiol. In addition, an aliquot of resin is removed and subjected to a both a Saville assay to quantitate S-nitrosothiol, and a DTNB assay to quantitate residual free thiol.

To trans-nitrosate glutathione-choline ester, the glutathione-choline ester (20 mM) is dissolved in DMF and added to the column. The column is capped and shaken for 5 minutes. The S-nitrosoglutathione choline ester is then eluted from the column, with any excess washed out using a small volume of DMF. The S-nitrosothiol and free thiol content of the S-nitrosoglutathione choline ester are then assayed using DTNB and Saville assays respectively, to gauge the efficiency of S-nitrosation. The resulting S-nitrosoglutathione choline ester can be further purified by HPLC, then lyophilized and stored at −80° C. In addition, the resin can be regenerated by removing any residual S-nitrosothiol (Cu²⁺, DTT and bright light), then re-reducing and re-nitrosating. Yield is quantified spectrophotometrically at 334 nm (Hogg (2002) Ann. Rev. Pharmacol. Toxicol. 42:585-600). Purity is determined by LC-MS analysis, and kinetics of NO^{•} release in biological systems are assayed using a NO^{•}-sensitive microelectrode (Dai, et al. (2001) Am. J. Physiol. 281:H2261-H2269).

Using similar methodologies, N-acetylcysteine choline ester and cysteine choline ester are S-nitrosated to afford S-nitroso-N-acetylcysteine choline ester 2 and S-nitroso-cysteine choline ester.

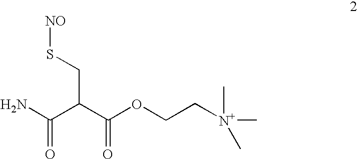

### EXAMPLE 12

### Preparation of S-Nitrosothiocarnitine

L-Carnitine and L-carnitine esters are transported into mitochondria by carnitine acetyltransferase. This transport system is highly selective and affords the use of carnitine-based prodrugs to deliver NO^{•} donor compounds to mitochondria. At least two synthetic routes can be used to prepare S-nitrosothiocarnitine.

L-(−)-Carnitine has an R absolute configuration (Englard, et al. (1985) Biochemistry 24:1110-1116; Kaneko & Yoshida (1962) Bull. Chem. Soc. Japan 35:1153-1155). Therefore, treatment of L-(−)-carnitine((R)-carnitine) with Lawesson's reagent affords (R)-thiocarnitine (Sheme 2), because the reaction proceeds with retention of configuration (Nishio (1993) J. Chem. Soc., Perkin Trans. 1:1113-1117). Nitrosation of (R)-carnitine affords (R)-S-nitrosothiocarnitine. The advantage of this route is that it makes available both enantiomers of thiocarnitine.

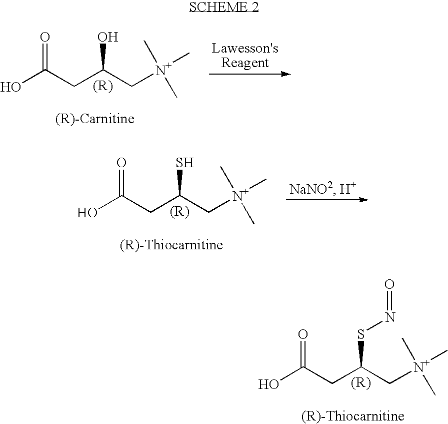

A second route includes the acid-catalyzed dehydration of carnitine to give 4-trimethylammonio-2-butenoic acid, followed by addition of thioacetic acid to give S-acetyl-DL-thiocarnitine (Scheme 3). Facile, base-catalyzed hydrolysis of the thioester and acidification in the presence of NaNO₂ gives S-acetyl-DL-thiocarnitine. An advantage of this route is that the intermediate S-acetyl-DL-thiocarnitine cannot undergo oxidation to the disulfide and could be stored and then rapidly converted to DL-thiocarnitine and, thence, to S-nitroso-DL-thiocarnitine 3.

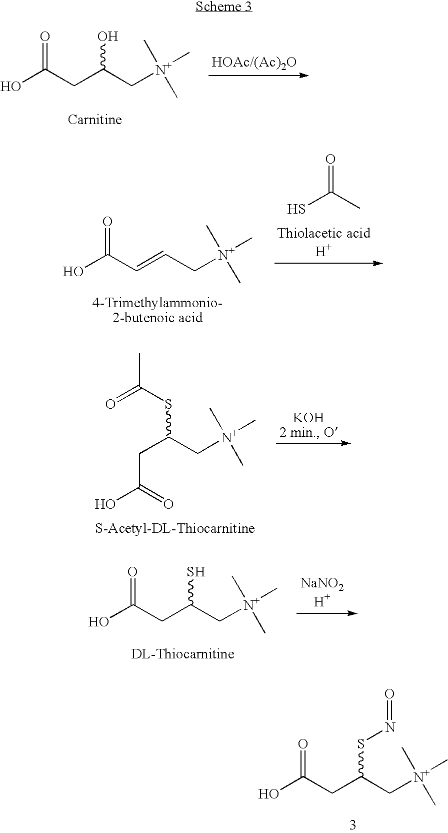

Advantageously, L-carnitine is absorbed from the intestinal tract by a Na⁺-dependent transporter, which may be the same as OCTN2 (Duran, et al. (2002) J. Membr. Biol. 185:65-74). Accordingly, (R)-S-nitrosothiocarnitine can effectively be used orally.

### EXAMPLE 13

### Preparation of S-Nitroso-2-Mercaptopropylglycine

2-Mercaptopropionylglycine 4, marketed as THIOLA™, is a known therapeutic agent approved for the treatment of bladder stones (Mission Pharmacal, San Antonio Tex.). 2-Mercaptopropionylglycine is has been described as a mitochondrial protective agent in ischemia-reperfusion injury (Tanonaka, et al. (2003) Cardiovas. Res. 57:416-425; Fuchs, et al. (1988) Arch. Biochem. Biophys. 266:83-88; Fuchs, et al. (1985) Basic Res. Cardiol. 80:231-240; Horwitz, et al. (1994) Circulation 89:1792-1801). However, very high concentrations of 2-mercaptopropionylglycine (0.3-1.0 mM) were required to elicit protection in cell and organ models of ischemia-reperfusion. 2-Mercaptopropionylglycine is bioavailable, and radioactive tracer studies in whole animals have demonstrated that it readily enters cells and accumulates in mitochondria (Chiba, et al. (1973) Yakugaku Zasshi 93:112-118.)

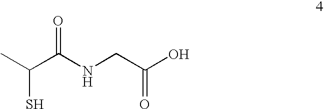

N-mercaptoalkanoyl cysteines are 2-mercaptopropionylglycine derivatives used to treat rheumatoid arthritis. Moreover, 3-mercapto-2-(2-mercapto-2-methylpropanamido)propanoic acid 5, also known as Bucillamine, has been suggested for use in ischemia-reperfusion injury (U.S. Pat. Nos. 5,756,547 and 5,670,545), but neither mention S-nitroso derivatives of the parent thiols.

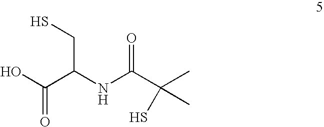

S-nitrosation of 2-mercaptopropionylglycine (and similar compounds) is readily carried out in accordance with the methods disclosed herein to afford S-nitroso-2-mercaptopropylglycine 6.

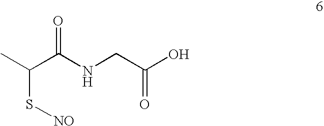

What is claimed is:

1. A S-nitrosated mitochondria-targeted thiol antioxidant prodrug.

2. A pharmaceutical composition comprising the prodrug of claim 1 in admixture with a pharmaceutically acceptable carrier.

3. A method for delivering nitric oxide to mitochondria of a cell comprising contacting a cell or tissue with a mitochondria-targeted thiol antioxidant prodrug of claim 1, thereby delivering nitric oxide to the mitochondria of the cell or cells of the tissue.

4. A method for decreasing the degree of mitochondrial dysfunction resulting from changes in the mitochondrial redox environment comprising contacting a cell or tissue with an effective amount of a mitochondria-targeted thiol antioxidant prodrug of claim 1, thereby decreasing the degree of mitochondrial dysfunction in the cell resulting from changes in the mitochondrial redox environment.

5. A method for preventing or treating a disease or condition associated with mitochondrial dysfunction resulting from changes in the mitochondrial redox environment comprising administering to a subject an effective amount of a pharmaceutical composition of claim 2 so that a disease or condition associated with mitochondrial dysfunction in the subject is prevented or treated.
